(19) United States
(12) Patent Application Publication
(10) Pub. No.: US 2025/0049696 A1
(43) Pub. Date: Feb. 13, 2025

(54) PHOTODIMERIZABLE POLYMERS COMPRISING AT LEAST ONE POLYOXYALKYLENE GROUP, COMPOSITION COMPRISING SAME AND COSMETIC TREATMENT PROCESS

(71) Applicant: L'Oreal, Paris (FR)

(72) Inventors: Constance Magne, CHEVILLY-LARUE (FR); Gabin Vic, CLICHY (FR); Eric Parris, SAINT OUEN (FR)

(73) Assignee: L'Oreal, Paris (FR)

(21) Appl. No.: 18/722,974

(22) PCT Filed: Dec. 21, 2022
(86) PCT No.: PCT/EP2022/087213

§ 371 Date: Jun. 21, 2024

(30) Foreign Application Priority Data
Dec. 23, 2021 (FR) FR2114451

(51) Int. Cl. A61K 8/91; A61Q 5/12; C08F 261/04

(52) U.S. Cl. CPC A61K 8/91; A61Q 5/12; C08F 261/04; A61K 2800/81

(57) ABSTRACT

The present invention relates to particular photodimerizable polymers comprising at least one polyoxyalkylene group, and also to a composition comprising at least one of these polymers. The present invention also relates to a cosmetic process for treating keratin materials, in particular keratin fibres, preferably human keratin fibres such as the hair, comprising the application to said keratin materials of at least one of these particular photodimerizable polymers.

The present invention relates to particular photodimerizable polymers comprising at least one polyoxyalkylene group, and also to a composition comprising at least one of these polymers.

The present invention also relates to a cosmetic process for treating keratin materials, in particular keratin fibres, preferably human keratin fibres such as the hair, comprising the application to said keratin materials of at least one of these particular photodimerizable polymers.

Hair is generally damaged and embrittled by the action of external atmospheric agents such as light and bad weather, and by mechanical or chemical treatments, such as brushing, combing, dyeing, bleaching, permanent-waving and/or relaxing.

Thus, in order to remedy these drawbacks, it is now common practice to pursue haircare treatments involving the use of care compositions that make it possible to condition the hair after these treatments in order notably to give it sheen, softness, suppleness, lightness, a natural feel and disentangling properties.

These haircare compositions may advantageously be compositions to be applied after shampooing and may be in the form of gels, hair lotions or more or less thick creams.

To improve the cosmetic properties of these compositions, it is known practice to introduce therein cosmetic agents, known as conditioning agents, intended mainly to repair or to limit the harmful or undesirable effects brought about by the various treatments or attacking factors to which hair fibres are more or less repeatedly subjected. These conditioning agents may, of course, also improve the cosmetic behaviour of natural hair.

With this aim, it has already been proposed to use cosmetically active organic compounds, such as cationic polymers and silicones, as conditioning agents in cosmetic care compositions, such as hair conditioners, in order to give the hair satisfactory cosmetic properties, in particular sheen, softness, suppleness, lightness, a natural feel and an improved ability to be disentangled.

However, the use of these compounds in care and/or conditioning cosmetic compositions does not afford the hair entirely satisfactory and lasting cosmetic properties. This is because these compositions generally provide cosmetic properties, such as the disentangling of wet and dry hair, suppleness, smoothness, sheen, coating and an individualized nature of the hair strands, which remain insufficient and which have a tendency to fade out after washing the hair with a standard shampoo.

Thus, there is a real need to provide novel compounds that are capable of remedying the abovementioned drawbacks, i.e. which are capable of providing, for all types of keratin materials, disentangling and conditioning properties which are not only satisfactory from the first application, but which also persist over time and withstand washing. These properties must also be homogeneous on all keratin materials, notably keratin fibres.

These aims are achieved by the present invention, the subject of which is in particular a photodimerizable (or photocrosslinkable or photosensitive) polymer (P) comprising at least one photodimerizable pendant group and at least one polyoxyalkylene pendant group, wherein the photodimerizable pendant group(s) are chosen from monovalent radicals of formulae (I) and (II) below:

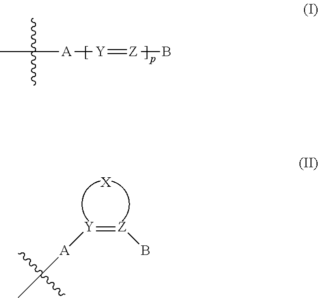

and also the geometrical isomers thereof,
in which formulae (I) and (II):
Y and Z denote, independently of each other, a nitrogen atom or a C(R) group with R representing a hydrogen atom or a (C₁-C₄)alkyl group such as methyl;
A represents a bond or a divalent group chosen from (C₁-C₈)alkylene, arylene, heteroarylene, cycloalkylene, heterocycloalkylene, (thio)carbonyl and (C₂-C₈)alkenylene radicals and combinations thereof;
B represents a monovalent group chosen from (C₁-C₈)alkyl radicals, aryl radicals, optionally cationic heteroaryl radicals, cycloalkyl radicals, optionally cationic heterocycloalkyl radicals, (thio)carbonyl radicals and (C₂-C₈)alkenyl radicals and combinations thereof,
X represents a divalent group chosen from (C₂-C₈)alkylene, arylene, heteroarylene, cycloalkylene, heterocycloalkylene, (thio)carbonyl and (C₂-C₈)alkenylene radicals and combinations thereof:
p represents an integer between 1 and 5 inclusive, in particular between 1 and 3; preferably, p is equal to 1;

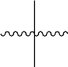

represents the bond which connects the part of the monovalent radical to the rest of the molecule; and
each of the groups mentioned may optionally be substituted with one or more halogen atoms or groups chosen from the following: (C₁-C₆)alkyl, hydroxyl, amino, (di)(C₁-C₆)alkylamino, phenyl, carboxyl, (C₁-C₆)alkoxy, (C₁-C₆)alkoxy(thio)carbonyl, hydrogeno(thio)carbonyl, sulfonato R—O—S(O)₂— or R—S(O)₂—O—, amide RR′N—C(O)— or R—C(O)—N(R′)— or acyl R—C(O)—, ammonium R′R″N⁺— with R, R′ and R″, which may be identical or different, representing a hydrogen atom or a (C₁-C₄)alkyl group.

The photodimerizable polymers (P) according to the invention make it possible to give good conditioning properties to keratin materials, in particular to keratin fibres, notably sheen, softness, suppleness, lightness, a natural feel, and also an improved ability to be disentangled.

Moreover, the conditioning properties afforded by means of the photodimerizable polymer according to the invention show good resistance to the various attacking factors to which hair may be subjected, such as light, bad weather, washing and perspiration. They are notably persistent with respect to shampoo washing.

A subject of the present invention is also a composition comprising one or more photodimerizable polymers (P) as defined previously.

The present invention also relates to a cosmetic process for treating keratin materials, in particular keratin fibres, preferably human keratin fibres such as the hair, comprising:
(a) a step of applying to said keratin materials one or more photodimerizable polymers (P), as defined previously,
(b) followed by an optional step of drying said keratin materials,
(c) followed by a step of exposing said keratin materials to natural or artificial light radiation.

Irradiation of the photodimerizable polymers (P) of the invention notably makes it possible to obtain a homogeneous and long-lasting deposit on all the keratin materials, which also has a good conditioning effect and which is suitable for all types of keratin materials. The materials thus treated have satisfactory or even improved conditioning properties, which persist over time and withstand washing. Hair strands in particular are easy to disentangle and have a soft, smooth feel.

Other subjects, features, aspects and advantages of the invention will emerge even more clearly on reading the description and the examples that follow.

In the text hereinbelow, and unless otherwise indicated:
the limits of a range of values are included in that range, notably in the expressions “between . . . and . . . ” and “ranging from . . . to . . . ”;
the expression “at least one” used in the present description is equivalent to the expression “one or more”;
the expression “keratin materials” denotes in particular the skin and keratin fibres, preferably human keratin fibres and more preferably the hair;
the term “alkylene chain” means an acyclic divalent hydrocarbon-based chain which is of C₁-C₂₀, particularly C₁-C₆, more particularly C₁-C₂ when the chain is linear, optionally substituted with one or more groups, which may be identical or different, chosen from i) hydroxyl, ii) (C₁-C₂)alkoxy, iii) (poly)hydroxy(C₂-C₄)alkoxy(di)(C₁-C₂)(alkyl)amino, iv) R^{a}—Z^{a}—C(Z^{b})—Z^{c}—, and v) R^{a}—Z^{a}—S(O)ₜ—Z^{c}— with Z^{a} and Z^{b}, which may be identical or different, representing an oxygen or sulfur atom, or a group NR^{a}′, Z^{c} representing a bond, an oxygen or sulfur atom, or a group NR^{a}; R^{a} representing an alkali metal, a hydrogen atom, an alkyl group, or alternatively is absent if another part of the molecule is cationic and R^{a}′ representing a hydrogen atom or an alkyl group; more particularly, the groups iv) are chosen from carboxylate —C(O)O⁻ or —C(O)OMetal (Metal=alkali metal), carboxyl —C(O)—OH, guanidino H₂H—C(NH₂)—NH—, amidino H₂H—C(NH₂)—, (thio)ureo H₂N—C(O)—NH— and H₂N—C(S)—NH—, aminocarbonyl —C(O)—NRa′₂ or aminothiocarbonyl —C(S)—NRa′₂; carbamoyl Ra′—C(O)—NRa′— or thiocarbamoyl Ra′—C(S)—NRa′— with Ra′, which may be identical or different, representing a hydrogen atom or a (C₁-C₄)alkyl group;
the term “(hetero)aryl” means aryl and heteroaryl groups;
the “aryl” or “heteroaryl” radicals or the aryl or heteroaryl part of a radical may be substituted with at least one substituent borne by a carbon atom, chosen from:
   a C₁-C₁₆ and preferably C₁-C₈ alkyl radical optionally substituted with one or more radicals chosen from hydroxyl, C₁-C₂ alkoxy, C₂-C₄ (poly)hydroxyalkoxy, acylamino, amino substituted with two C₁-C₄ alkyl radicals, which may be identical or different, optionally bearing at least one hydroxyl group, or the two radicals possibly forming, with the nitrogen atom to which they are attached, a saturated or unsaturated, optionally substituted 5- to 7-membered and preferably 5- or 6-membered heterocycle optionally comprising another nitrogen or non-nitrogen heteroatom;
   a halogen atom such as chlorine;
   a hydroxyl group;
   a C₁-C₂ alkoxy radical;
   a (poly)hydroxy(C₂-C₄)alkoxy radical;
   an amino radical;
   a 5- or 6-membered heterocycloalkyl radical;
   an optionally cationic 5- or 6-membered heteroaryl radical, preferentially imidazolium, optionally substituted with a (C₁-C₄)alkyl radical, preferentially methyl;
   an amino radical substituted with one or two identical or different C₁-C₆ alkyl radicals, optionally bearing at least:
i) a hydroxyl group,
ii) an amino group optionally substituted with one or two optionally substituted C₁-C₃ alkyl radicals, said alkyl radicals possibly forming with the nitrogen atom to which they are attached a saturated or unsaturated, optionally substituted 5- to 7-membered heterocycle, optionally comprising at least one other nitrogen or non-nitrogen heteroatom,
iii) a quaternary ammonium group —N⁺R′R″R′″, M- for which R′, R″ and R′″, which may be identical or different, represent a hydrogen atom or a C₁-C₄ alkyl group; and M- represents the counterion of the corresponding organic or mineral acid or of the corresponding halide;
iv) or an optionally cationic 5- or 6-membered heteroaryl radical, preferentially imidazolium, optionally substituted with a (C₁-C₄)alkyl radical, preferentially methyl;
   an acylamino radical (—N(R)—C(O)—R′) in which the radical R is a hydrogen atom or a C₁-C₄ alkyl radical optionally bearing at least one hydroxyl group and the radical R′ is a C₁-C₂ alkyl radical;
   a carbamoyl radical ((R)₂N—C(O)—) in which the radicals R, which may be identical or different, represent a hydrogen atom or a C₁-C₄ alkyl radical optionally bearing at least one hydroxyl group;
   an alkylsulfonylamino radical (R′—S(O)₂—N(R)—) in which the radical R represents a hydrogen atom or a C₁-C₄ alkyl radical optionally bearing at least one hydroxyl group and the radical R′ represents a C₁-C₄ alkyl radical, or a phenyl radical;
   an aminosulfonyl radical ((R)₂N—S(O)₂—) in which the radicals R, which may be identical or different, represent a hydrogen atom or a C₁-C₄ alkyl radical optionally bearing at least one hydroxyl group;
   a carboxylic radical in acid or salified (preferably with an alkali metal or a substituted or unsubstituted ammonium) form;
   a cyano group;
   a nitro or nitroso group;
   a polyhaloalkyl group, preferentially trifluoromethyl;
the cyclic, cycloalkyl or heterocyclic part of a non-aromatic radical may be substituted with at least one substituent chosen from the following groups:
   hydroxyl;
   C₁-C₄ alkoxy, (poly)hydroxy(C₂-C₄)alkoxy;
   C₁-C₄ alkyl;
   alkylcarbonylamino (R—C(O)—N(R′)—) in which the radical R′ is a hydrogen atom or a C₁-C₄ alkyl radical optionally bearing at least one hydroxyl group, and the radical R is a C₁-C₂ alkyl radical or an amino radical optionally substituted with one or two C₁-C₄ alkyl groups, which may be identical or different, themselves optionally bearing at least one hydroxyl group, said alkyl radicals possibly forming, with the nitrogen atom to which they are attached, a saturated or unsaturated, optionally substituted 5- to 7-membered heterocycle optionally comprising at least one other nitrogen or non-nitrogen heteroatom;
   alkylcarbonyloxy (R—C(O)—O—) in which the radical R is a C₁-C₄ alkyl radical or an amino group optionally substituted with one or two identical or different C₁-C₄ alkyl groups themselves optionally bearing at least one hydroxyl group, said alkyl radicals possibly forming with the nitrogen atom to which they are attached a saturated or unsaturated, optionally substituted 5- to 7-membered heterocycle, optionally comprising at least one other nitrogen or non-nitrogen heteroatom;
   alkoxycarbonyl (R-G-C(O)—) in which the radical R is a C₁-C₄ alkoxy radical, G is an oxygen atom or an amino group optionally substituted with a C₁-C₄ alkyl group optionally bearing at least one hydroxyl group, said alkyl radical possibly forming with the nitrogen atom to which they are attached a saturated or unsaturated, optionally substituted 5- to 7-membered heterocycle, optionally comprising at least one other nitrogen or non-nitrogen heteroatom;
a cyclic, cycloalkyl or heterocyclic radical, or a non-aromatic part of an aryl or heteroaryl radical, may also be substituted with one or more oxo groups;
a cycloalkyl radical is a monocyclic or bicyclic hydrocarbon-based radical, comprising from 3 to 10 carbon atoms and particularly from 4 to 7 carbon atoms, such as cyclopentyl or cyclohexyl;
a hydrocarbon-based chain is unsaturated when it includes one or more double bonds and/or one or more triple bonds;
an “aryl” radical represents a monocyclic or fused or non-fused polycyclic carbon-based group comprising from 6 to 22 carbon atoms, and in which at least one ring is aromatic; preferentially, the aryl radical is a phenyl, biphenyl, naphthyl, indenyl, anthracenyl or tetrahydronaphthyl;
a “heteroaryl radical” represents a 5- to 22-membered, monocyclic or fused or non-fused polycyclic group, comprising from 1 to 6 heteroatoms chosen from nitrogen, oxygen, sulfur and selenium atoms, at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridinyl, tetrazolyl, dihydrothiazolyl, imidazopyridyl, imidazolyl, indolyl, isoquinolyl, naphthoimidazolyl, naphthoxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridinyl, thiazoylimidazolyl, thiopyrylyl, triazolyl, xanthyl and the ammonium salt thereof;
a “heterocyclic radical” is a 5- to 22-membered monocyclic or fused or non-fused polycyclic radical which may contain one or two unsaturations but is non-aromatic, including from 1 to 6 heteroatoms chosen from nitrogen, oxygen, sulfur and selenium;
a “heterocycloalkyl radical” is a heterocyclic radical comprising at least one saturated ring;
a “cationic heteroaryl radical” is a heteroaryl group as defined previously, which includes an endocyclic or exocyclic cationic group;
when the charge is endocyclic, it is included in the electron delocalization via the mesomeric effect; for example, it is a pyridinium, imidazolium or indolinium group:

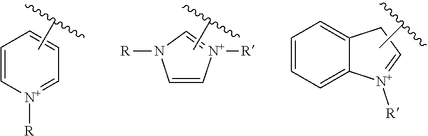

with R and R′ being a heteroaryl substituent as defined previously and particularly a (hydroxy)(C₁-C₈)alkyl group, such as methyl;
   when the charge is exocyclic, it is not included in the electron delocalization via the mesomeric effect; for example, it is an ammonium or phosphonium substituent R⁺, such as trimethylammonium, which is outside the heteroaryl, such as pyridyl, indolyl, imidazolyl or naphthalimidyl, in question:

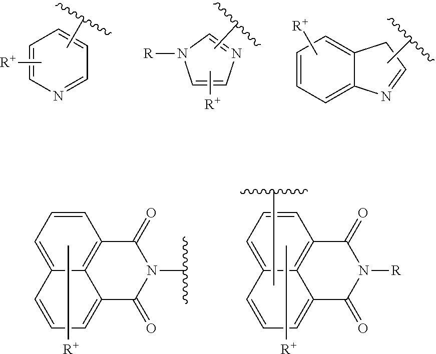

with R being a heteroaryl substituent as defined below and R⁺ an ammonium RₐR_{b}R_{c}N⁺—, phosphonium RₐR_{b}R_{c}P⁺— or ammonium RₐR_{b}R_{c}N⁺—(C₁-C₆)alkylamino, RₐR_{b}R_{c}N⁺—(C₁-C₆)alkyl or RₐR_{b}R_{c}N⁺—(C₁-C₆)alkoxy group with Rₐ, R_{b} and R_{c}, which may be identical or different, representing a (C₁-C₈)alkyl group such as methyl;
   a “cationic aryl bearing an exocyclic charge” means an aryl ring whose quaternized cationic group is outside said ring: it is notably an ammonium or phosphonium substituent R⁺ such as trimethylammonium, outside the aryl such as phenyl or naphthyl:

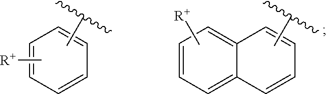

an “alkyl radical” is a linear or branched C₁ to C₂₀, preferably C₁ to C₁₀, more preferentially C₁ to C₈, better still C₁ to C₆ and even better still C₁ to C₄ hydrocarbon-based radical;
an “alkenylene radical” is an unsaturated divalent hydrocarbon-based radical as defined previously, which may contain from 1 to 4 conjugated or unconjugated double bonds —C═C—; the alkenylene group particularly contains 1 or 2 unsaturations;
the expression “optionally substituted” applied to the alkyl radical implies that said alkyl radical may be substituted with one or more radicals chosen from the following radicals: i) hydroxyl, ii) C₁-C₄ alkoxy, iii) R—Z—C(X)—Y— with X, Y and Z representing an oxygen or sulfur atom or N(R′), or alternatively X and/or Z represent a bond, R and R′, which may be identical or different, represent a hydrogen atom or a (C₁-C₆)alkyl group, preferably, X represents an oxygen atom, iv) amino optionally substituted with one or two identical or different C₁-C₄ alkyl radicals, said alkyl radicals possibly forming, with the nitrogen atom that bears them, a 5- to 7-membered heterocycle, optionally comprising another nitrogen or non-nitrogen heteroatom; v) a quaternary ammonium group N⁺R′R″R′″, M- for which R′, R″ and R′″, which may be identical or different, represent a C₁-C₄ alkyl group, or alternatively —N⁺R′R″R′″ forms a heteroaryl such as imidazolium optionally substituted with a C₁-C₄ alkyl group and M- represents the anionic counterion, vi) carboxyl C(O)OH, vii) carboxylate C(O)O⁻, M⁺ with M⁺ representing a cationic counterion such as alkali metal or alkaline-earth metal, viii) sulfonic —SO₃H, ix) sulfonate —SO₃—, M⁺ with M⁺ as defined previously, x) cyano and xi) phenyl;
an “alkoxy radical” is an alkyl-oxy radical for which the alkyl radical is a linear or branched C₁-C₁₆ and preferentially C₁-C₈ hydrocarbon-based radical; when the alkoxy group is optionally substituted, this implies that the alkyl group is optionally substituted as defined previously;
the term “organic or mineral acid salt” more particularly means salts chosen from a salt derived from i) hydrochloric acid HCl, ii) hydrobromic acid HBr, iii) sulfuric acid H₂SO₄, iv) alkylsulfonic acids: Alk-S(O)₂OH such as methanesulfonic acid and ethanesulfonic acid; v) arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk-O—S(O)—OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) acetic acid CH₃C(O)—OH; xiv) triflic acid CF₃SO₃H; and xv) tetrafluoroboric acid HBF₄;
the term “anionic counterion” means an anion or an anionic group derived from an organic or mineral acid which counterbalances the cationic charge of the compound; more particularly, the anionic counterion is chosen from: i) halides such as chloride or bromide; ii) nitrates; iii) sulfonates, including C₁-C₆ alkylsulfonates: Alk-S(O)₂O⁻ such as methylsulfonate or mesylate and ethylsulfonate; iv) arylsulfonates: Ar—S(O)₂O⁻ such as benzenesulfonate and toluenesulfonate or tosylate; v) carboxylates Alk-C(O)—OH with Alk representing a (C₁-C₆)alkyl group optionally substituted with one or more hydroxyl or carboxylate groups such as citrate; vi) succinate; vii) tartrate; viii) lactate; ix) alkyl sulfates: Alk-O—S(O)O⁻ such as methyl sulfate and ethyl sulfate; x) aryl sulfates: Ar—O—S(O)O⁻ such as benzene sulfate and toluene sulfate; xi) alkoxy sulfates: Alk-O—S(O)₂O⁻ such as methoxy sulfate and ethoxy sulfate; xii) aryloxy sulfates: Ar—O—S(O)₂O⁻, xiii) phosphates O═P(OH)₂—O⁻, O═P(O⁻)₂—OH O═P(O⁻)₃, HO—[P(O)(O⁻)]_{w}—P(O)(O⁻)₂ with w being an integer; xiv) acetate; xv) triflate; and xvi) borates such as tetrafluoroborate, and xvii) sulfate (O═)₂S(O⁻)₂ or SO₄²⁻ and hydrogen sulfate HSO₄⁻;
the anionic counterion, derived from the organic or mineral acid salt, ensures the electrical neutrality of the molecule; thus, it is understood that when the anion comprises several anionic charges, then the same anion may serve for the electrical neutrality of several cationic groups in the same molecule or else may serve for the electrical neutrality of several molecules; for example, a dye which contains two cationic groups may contain either two “singly charged” anionic counterions or a “doubly charged” anionic counterion such as (O═)₂S(O⁻)₂ or O═P(O⁻)₂—OH;
   moreover, the addition salts that may be used in the context of the invention are notably chosen from addition salts with a cosmetically acceptable base, such as the basifying agents as defined below, for instance alkali metal hydroxides such as sodium hydroxide, potassium hydroxide, aqueous ammonia, amines or alkanolamines.

For the purposes of the present invention, the term “photodimerizable group” means a chemical group that leads to photodimerization reactions under irradiation, i.e. after exposure to natural or artificial light radiation. In the context of the invention, photodimerization is a chemical reaction between two double bonds (of 2+2 type) or two pairs of double bonds (of 4+4 type), and more particularly between two double bonds (of 2+2 type).

The case of a reaction between two double bonds may be represented schematically in the following manner:

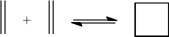

These photodimerization reactions are defined in the book “Advanced Organic Chemistry”, J. March, 4th edition, Wiley Interscience, NY 1992, page 855.

Thus, the double bond, when it is photo-stimulated, generally when it is subjected to UV radiation, proves to be capable of reacting with another double bond by cyclization. According to the present invention, the double bond is said to be activated, i.e. it is spontaneously photodimerizable, without requiring the presence of a photoinitiator or a chemical initiator.

This double bond is generally activated by the presence of an electron-withdrawing substituent in the alpha position of this photodimerizable double bond.

As electron-withdrawing substituent, mention may be made of aromatic nuclei such as the phenyl group optionally substituted with one or more halogen atoms, or electron-withdrawing groups such as NO₂, CN, R′—Y—C(Y′)—, R′—C(Y′)—Y—, R′—Y—C(Y′)—Y—, R′—Y—S(O)₂— or —S(O)₂—Y—R′, where R′ represents a hydrogen atom or a (C₁-C₄)alkyl group optionally substituted with one or more halogen atoms, where Y and Y′, which may be identical or different, represent an oxygen or sulfur atom or NR″ where R″ represents a hydrogen atom or a (C₁-C₆)alkyl group.

### Photodimerizable Polymers (P)

The present invention relates to a photodimerizable polymer (P) comprising at least one photodimerizable pendant group and at least one polyoxyalkylene pendant group.

The terms “photodimerizable”, “photocrosslinkable” and “photosensitive” are equivalent within the meaning of the present invention.

The photodimerizable pendant groups used according to the invention are chosen from the monovalent radicals of formulae (I) and (II) below:

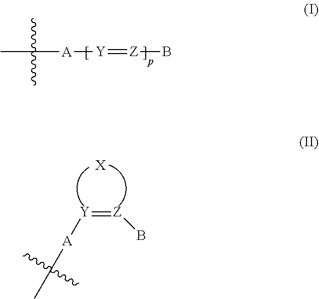

and also the geometrical isomers thereof,
in which formulae (I) and (II):
   Y and Z denote, independently of each other, a nitrogen atom or a C(R) group with R representing a hydrogen atom or a (C₁-C₄)alkyl group such as methyl;
   A represents a bond or a divalent group chosen from (C₁-C₈)alkylene, arylene, heteroarylene, cycloalkylene, heterocycloalkylene, (thio)carbonyl and (C₂-C₈)alkenylene radicals and combinations thereof,
   B represents a monovalent group chosen from (C₁-C₈)alkyl radicals, aryl radicals, optionally cationic heteroaryl radicals, cycloalkyl radicals, optionally cationic heterocycloalkyl radicals, (thio)carbonyl radicals and (C₂-C₈)alkenyl radicals and combinations thereof,
   X represents a divalent group chosen from (C₂-C₈)alkylene, arylene, heteroarylene, cycloalkylene, heterocycloalkylene, (thio)carbonyl and (C₂-C₈)alkenylene radicals and combinations thereof,
   p represents an integer between 1 and 5 inclusive, in particular between 1 and 3; preferably, p is equal to 1;

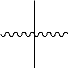

represents the bond which connects the part of the monovalent radical to the rest of the molecule; and
each of the groups mentioned may optionally be substituted with one or more halogen atoms or groups chosen from the following: (C₁-C₆)alkyl, hydroxyl, amino, (di)(C₁-C₆)alkylamino, phenyl, carboxyl, (C₁-C₆)alkoxy, (C₁-C₆)alkoxy(thio)carbonyl, hydrogeno(thio)carbonyl, sulfonato R—O—S(O)₂— or R—S(O)₂—O⁻, amide RR′N—C(O)— or R—C(O)—N(R′)— or acyl R—C(O)—, ammonium R′R″N⁺— with R, R′ and R″, which may be identical or different, representing a hydrogen atom or a (C₁-C₄)alkyl group.

The dimerizable pendant groups according to the invention are notably those mentioned in patents US-2 811 510, EP 0 313 220, EP 0 313 221, EP 092 901, GB 2 030 575 and GB 2 076 826, and also in the articles “Chemical Review Vol. 83, 5, 1983, page 507” “Polym, Paint Colour Journal 1988, 178, page 209” and “Current Trends in Polymer Photochemistry, Ellis Morwood edition, NY, 1995”.

More preferentially, the photodimerizable pendant group(s) are chosen from the monovalent radicals of the following compounds:
stilbene,
styrylpyridinium (stilbazolium) of formulae (A1) and (A2) below, and the geometrical isomers thereof:

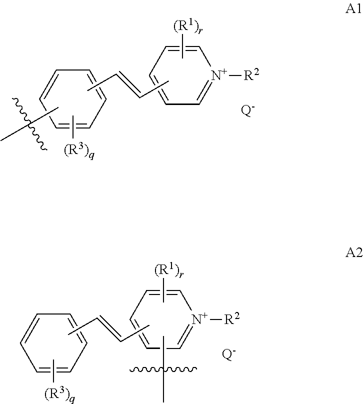

in which:
   R¹ and R³, which may be identical or different, represent a halogen atom or a (C₁-C₆)alkyl group; or else two contiguous R¹ or R³ groups form, together with the carbon atoms bearing them, a benzo group;
   R² represents a hydrogen atom, a (C₁-C₆)alkyl group optionally substituted with one or more halogen atoms such as chlorine or hydroxyl; preferably, R² represents a (C₁-C₆)alkyl group such as methyl, ethyl or propyl;
   q and r represent an integer between 0 and 4 inclusive; and
   Q⁻ represents an anionic counter ion preferably chosen from halide ions such as chlorides, bromides and iodides, perchlorates, tetrafluoroborates, methyl sulfate, phosphates, sulfates, methanesulfonates, p-toluenesulfonate; and

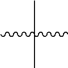

represents the bond which connects the part of the monovalent radical to the rest of the molecule, it being understood that the pendant group A2 can be connected to the rest of the molecule via R²;
preferably, the

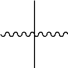

bond is on the phenyl in the para position of the styryl group on A1 or connected to the rest of the molecule via R² on A2; preferentially, the styryl group of A1 and A2 is in the para position of the pyridinium group;
styrylazolium of formulae (B1 and B2) below, and the geometrical isomers thereof:

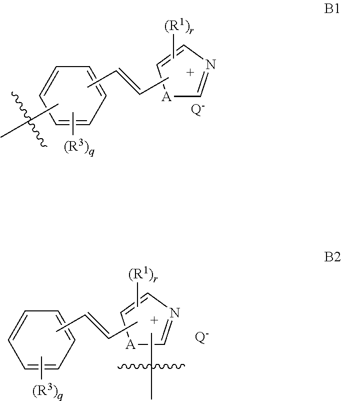

in which:
   A represents a sulfur atom, an oxygen atom, or a group NR₂ or C(R²)₂; and

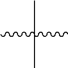

Q⁻, r, q, R¹, R² and R³ being as defined previously,
preferably, the

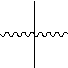

bond is on the phenyl in the para position of the stryryl group,
styrylpyrazine,
chalcone,
(thio)cinnamate and (thio)cinnamamide,
maleimide,
(thio)coumarin,
thymine,
uracil,
butadiene,
anthracene,
pyridone,
pyrrolizinone,
acridizinium salts,
furanone,
phenylbenzoxazole, and
derivatives thereof.

According to a particular embodiment, the photodimerizable pendant group(s) are chosen from:
a) photodimerizable group(s) bearing a stilbazolium function of formula (Ia) or (Ib) and the geometrical isomers thereof:

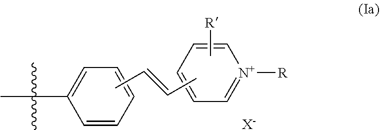

in which:
   R represents a hydrogen atom or a C₁-C₄ alkyl or C₁-C₄ hydroxyalkyl group;
   R′ represents a hydrogen atom or a C₁-C₄ alkyl group; and
   X⁻ denotes an anionic counterion preferably chosen from halide ions such as chlorides, bromides and iodides, perchlorates, tetrafluoroborates, methyl sulfate, phosphates, sulfates, methanesulfonates and p-toluenesulfonate; preferably, the styryl group is located para to the pyridinium group and/or para to the

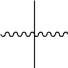

bond;

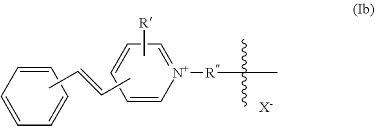

in which:
   R″ denotes a divalent alkylene radical containing from 2 to 8 carbon atoms;
   R′ represents a hydrogen atom or a C₁-C₄ alkyl group; and
   X⁻ having the same meaning as that described for the preceding formula (Ia);

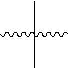

having the same meaning as previously;
preferably, the styryl group is located para to the pyridinium group;
or
b) photodimerizable groups bearing a styrylazolium function of formula (IIa):

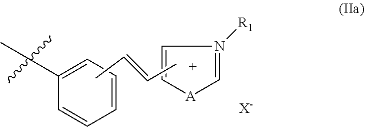

in which:
   R₁ denotes a hydrogen atom or a C₁-C₄ alkyl or C₁-C₄ hydroxyalkyl group;
   A denotes a sulfur atom, an oxygen atom or a group NR′ or C(R′)₂, R′; with R′ representing a hydrogen atom or a C₁-C₄ alkyl, R′ preferably representing a hydrogen atom; and
   X⁻ having the same meaning as that described for the preceding formula (Ia);

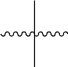

having the same meaning as previously;
preferably, the styryl group is located on the phenyl para to the bond

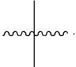

Such photodimerizable pendant groups bear activated double bonds, and as such the photodimerization of these double bonds starts spontaneously in the UVA range, without requiring a photoinitiator.

For the purposes of the present invention, the term “photoinitiator” means a compound which initiates the photodimerization reaction and releases a radical under irradiation, notably in the UV range.

The photodimerizable polymer(s) (P) according to the present invention also include one or more polyoxyalkylene pendant groups.

Preferably, the polyoxyalkylene pendant group(s) comprise one or more polyoxyalkylene groups chosen from the groups of the following formula:

—[O—X]ₙ—OR
in which, X represents an alkylene group containing from 2 to 3 carbon atoms, preferably 2 carbon atoms, n denotes an integer ranging from 10 to 1000, preferably from 20 to 200, and more preferentially from 25 to 150, and R represents a linear or branched alkyl chain containing from 1 to 6 carbon atoms.

According to a first embodiment, the polyoxyalkylene pendant group(s) of the photodimerizable polymers (P) comprise one or more polyoxyethylene group(s) corresponding to the formula —[O—CH₂—CH₂]ₙ—OR, with n denoting an integer ranging from 20 to 500, preferably from 30 to 150, and R representing a linear or branched alkyl chain containing from 1 to 6 carbon atoms. In other words, according to this embodiment, the photodimerizable polymer(s) (P) comprise one or more PEG (polyethylene glycol) groups. More particularly, use will be made of photodimerizable polymers (P) comprising one or more polyoxyethylene groups, corresponding to the formula —[O—CH₂—CH₂]ₙ—OR, with n denoting an integer ranging from 40 to 50 or from 120 to 150, and R representing a linear or branched alkyl chain containing from 1 to 6 carbon atoms.

According to another embodiment, the polyoxyalkylene pendant group(s) of the photodimerizable polymers (P) comprise one or more linear or branched polyoxypropylene groups, preferably corresponding to the formula —[O—CH₂—CH₂—CH₂]ₙ—OR, with n denoting an integer ranging from 10 to 500, preferably from 30 to 200, and R representing a linear or branched alkyl chain containing from 1 to 6 carbon atoms. In other words, according to this embodiment, the photodimerizable polymer(s) (P) comprise one or more PPG (polypropylene glycol) groups.

The photodimerizable polymer(s) (P) include one or more polyoxyalkylene pendant groups preferably comprising from 30 to 150 polyoxyethylene groups.

The backbone of the photodimerizable polymer(s) (P) according to the invention may be of various kinds. This polymer backbone may be natural or synthetic.

As natural polymer backbones, mention may be made of polysaccharides.

As polysaccharides, mention may be made of xanthan, carrageenan, chitosan, cellulose and its derivatives, alginate, starch, dextran, pullulan, galactomannan and the biologically acceptable salts thereof, and derivatives thereof.

Synthetic backbones that may be mentioned include polyvinyl alcohols, poly(vinyl) polymers and polydiorganosiloxanes.

Among the poly(vinyl) polymers, mention may be made of partially or totally hydrolysed poly(vinyl acetate)s, and polyvinyl alcohol (PVA).

The photodimerizable polymer(s) (P) of the invention have a natural or synthetic backbone preferably chosen from polysaccharides, poly(vinyl) polymers and polydiorganosiloxanes, and more preferentially from polyvinyl alcohols and poly(vinyl acetate)s, which are preferably partially hydrolysed.

As regards the photodimerizable polymers (P) containing photodimerizable pendant groups bearing a stilbazolium function, they are obtained by reaction of the polymer under consideration with a chemical species including a group of formula (Ia) or (Ib).

Preferably, the chemical species including a group (Ia) bears a reactive group W of aldehyde or acetal type.

As chemical species that may be used for grafting styrylpyridinium groups, mention may notably be made of quaternary salts of 2-(4-formylstyryl)pyridinium, 4-(4-formylstyryl)pyridinium, 2-(3-formylstyryl)pyridinium, N-methyl-2-(4-formylstyryl)pyridinium, N-methyl-3-(4-formylstyryl)pyridinium, N-methyl-2-(3-formylstyryl)pyridinium, N-methyl-2-(2-formylstyryl)pyridinium, N-ethyl-2-(4-formylstyryl)pyridinium, N-(2-hydroxyethyl)-2-(4-formylstyryl)pyridinium, N-(2-hydroxyethyl)-4-(4-formylstyryl)pyridinium, N-methyl-4-(4-formylstyryl)pyridinium or N-methyl-4-(3-formylstyryl)pyridinium.

The pyridinium quaternary salts may be chloride, bromide, iodide, perchlorate, tetrafluoroborate, methosulfate, phosphate, sulfate, methanesulfonate or p-toluenesulfonate salts. Such chemical species are described in GB-A-2030575.

Examples of chemical species that may be mentioned include 4-(4-formylphenylethenyl)-1-methylpyridiunium methosulfate, 1-(3-ethoxycarbonylmethyl)-4-[2-(4-formylphenyl)ethenyl]pyridinium bromide and 1-(methoxycarbonylpropyl)-4-[2-(4-formylphenyl)ethenyl]pyridinium bromide. Such species are described in US 2007/0112094.

Use is preferably made of n-methyl-4-(4-formylstyryl)pyridinium methyl sulfate (RN=74401-04-0), notably sold by the company Wako.

The synthesis, described below, of these polymers functionalized with photodimerizable groups such as those comprising a styryl group and polyoxyalkylene groups may be performed on the basis of the protocol of T. Uhlich et al. (Reactive & Functional Polymers, 28, 55-40 (1995)).

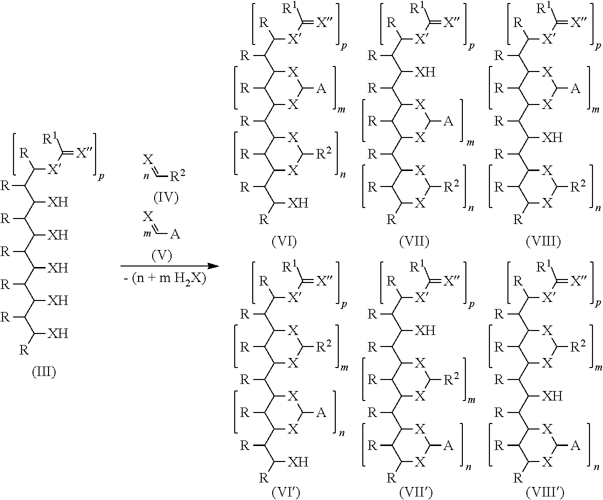

in which compounds of formulae (III) to (VIII′)
R, which may be identical or different, represents a hydrogen atom or a C₁-C₁₀ alkyl group, optionally substituted and/or interrupted with one or more heteroatoms; preferably, R represents a hydrogen atom or a (C₁-C₄)alkyl group such as methyl, ethyl or propyl; more preferentially, R represents a hydrogen atom;
R¹ represents a hydrogen atom or a (C₁-C₁₀)alkyl group, optionally substituted and/or interrupted with one or more heteroatoms; preferably, R¹ represents a hydrogen atom or a (C₁-C₄)alkyl group;
R² represents a polyoxyalkylene group of the following formula: —[O—X]ₙ—OR
in which, X represents an alkylene group containing from 2 to 3 carbon atoms, preferably 2 carbon atoms, n denotes an integer ranging from 10 to 1000, preferably from 20 to 200, and more preferentially from 25 to 150, and R represents a linear or branched alkyl chain containing from 1 to 6 carbon atoms;
A represents a group derived from a photodimerizable compound, preferably a styrylpyridinium compound such as (I), (II), (A1), (A2), (Ia), (Ib) or (IIa) as defined previously, more particularly chosen from (A1) or (Ia) as defined previously;
X represents an oxygen or sulfur atom, preferably oxygen;
X′ and X″ represent an oxygen or sulfur atom or a group N(R³) with R³ representing a hydrogen atom or a (C₁-C₄)alkyl group; preferably, X′ and X″ represent an oxygen atom.

Most of the products obtained are of formula (VII′).

Advantageously, these chemical species react with a polymer of polyvinyl alcohol or polyvinyl acetal type as described in the documents mentioned previously and also such as the polymer (III) described in the above scheme for which X, X′ and X″ represent an oxygen atom, R and R¹ being as described previously.

For example, a polyvinyl alcohol graft polymer comprising units of the following structure is thus obtained, A representing a group (I), (A1) or (Ia):

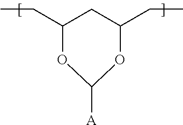

Polyvinyl alcohol polymers grafted with a styrylpyridinium group are notably described in the publication Ichimura K. et al., Preparation and characteristics of photo-crosslinkable poly(vinyl alcohol), Journal of Polymer Science, Polymer Chemistry Edition, Vol. 20, 1419-1432 (1982).

The polymers may be obtained by reacting polyvinyl alcohol or partially hydrolysed polyvinyl acetate with styrylpyridinium salts bearing a formyl or acetal group as described in GB-A-2 030 575, WO 96/29312, U.S. Pat. No. 5,061,603, GB-A-2076826 or EP-A-092901.

Cellulose polymers grafted with styrylpyridinium groups are notably described in US 2007/0 112 094.

Preferably, the chemical species including a group (A1) or (Ia) bears a reactive group which is a halogen atom such as chlorine.

In this embodiment, the chemical species has, for example, the formula

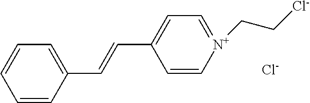

Advantageously, the photodimerizable polymer comprising the groups (Ib) is obtained, for example, by reaction of the above species with the polysaccharide chosen from those defined previously.

As regards the photodimerizable polymers containing photodimerizable groups bearing a styrylazolium function, they are obtained by reaction of the polymer with a chemical species including a group of formula (IIa).

Preferably, the chemical species including a group (IIa) bears a reactive group W of aldehyde or acetal type.

As chemical species that may be used for grafting groups of styrylazolium type, mention may be made of those described in EP-A-313 220.

Advantageously, these chemical species react with a polymer of polyvinyl alcohol or polyvinyl acetate type as described in the documents mentioned previously.

A polyvinyl alcohol grafted polymer comprising units having the following structure is thus obtained, with B corresponding to the group

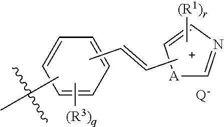

or (IIa) as defined previously:

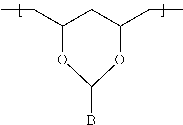

Polyvinyl alcohol polymers grafted with styrylazolium groups are notably described in EP-A-313 220. In said document, these polymers may be obtained by reaction of polyvinyl alcohol or partially hydrolysed polyvinyl acetate with styrylazolium salts bearing an aldehyde or acetal group.

According to one embodiment, the photodimerizable polymer (P) of the invention is in the form of particles, in particular dispersed particles. Thus, in the latter case, the polymer particles are more particularly polyvinyl alcohol particles.

According to a preferred embodiment, the photodimerizable polymer (P) of the invention is soluble in the cosmetic medium.

Thus, according to one embodiment variant, the photodimerizable polymer (P) is a polyvinyl acetate (PVA) type polymer partly functionalized with one or more polyoxyalkylene functions and one or more functions of formula (IX):

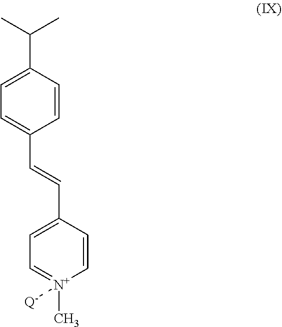

with Q⁻ being as defined previously.

The degree of polymerization of the PVA may be between 100 and 5000 and the degree of substitution, as a percentage of the functions of formula (I) as defined above, may be between 0.1% and 25%, preferably between 0.5% and 5%.

The following diagram represents a variant in which the photodimerizable polymer (P) is the polymer (III) as defined previously bearing functions grafted with stilbazolium species such as those of formula (A1) as defined previously, which is capable of crosslinking under the effect of light, as illustrated below.

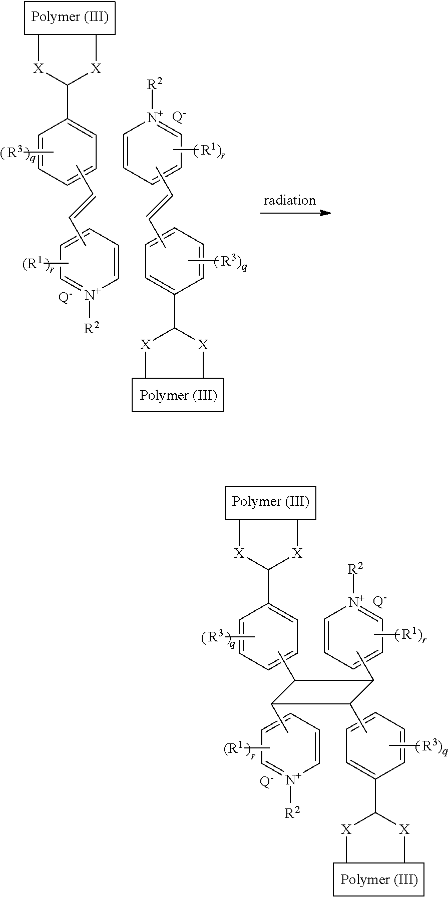

These materials react to radiation that may include both a UV light and visible light component, particularly a low dose of UV.

Preferentially, the following scheme represents the mPEG-PVA-SbQ polymer (PV Acetate polymer bearing polyoxyethylene functions and functions grafted with stilbazolium species), which is capable of crosslinking under the effect of light, as illustrated below.

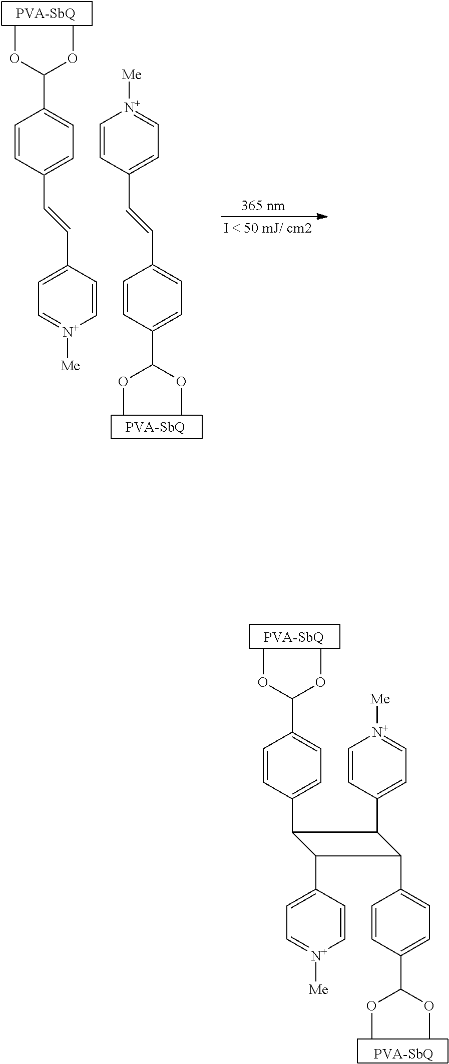 [SMILES: COC(OC)c1ccc(/C=C/c2cc[n+](C)cc2)cc1 ; COC(OC)c1ccc(/C=C/c2cc[n+](C)cc2)cc1 ; COC(OC)c1ccc(C2C(c3cc[n+](C)cc3)C(c3ccc(C(OC)OC)cc3)C2c2cc[n+](C)cc2)cc1]

These materials are particularly appreciated since they do not require a photoinitiator and react with radiation that may include both UV light and visible light, in particular a low dose of UV.

Pendant groups which have reactivity both in UV light and in visible light are favoured.

According to another embodiment variant, the photodimerizable polymer (P) is represented by a natural polymer which is functionalized with photodimerizable groups and polyoxyalkylene groups.

It may notably be a polysaccharide that may notably be chosen from chondroitin sulfate, keratan, keratan sulfate, heparin, heparin sulfate, xanthan, carrageenan, hyaluronic acid, chitosan, cellulose and derivatives thereof, alginate, starch, dextran, pullulan, galactomannan and biologically acceptable salts thereof.

Needless to say, the degree of functionalization is adjusted be able to provide the degree of crosslinking required during activation.

According to the invention, the degree of functionalization with photodimerizable units is at least 0.1%, or even at least 0.5%, or even at least 1%.

The total amount of the photodimerizable pendant group(s) present in the photodimerizable polymer (P) according to the invention is preferably from 0.5 to 6 mol %, and more preferentially from 1 to 5 mol %, relative to the total molar weight of the polymer.

The total amount of the polyoxyalkylene pendant group(s) present in the photodimerizable polymer (P) according to the invention preferably ranges from 0.1 to 5 mol %, and more preferentially from 0.5 to 3 mol %, relative to the total molar weight of the polymer.

The photodimerizable polymer (P) according to the invention preferably has a number-average molecular weight ranging from 2000 to 200 000, more preferentially from 5000 to 100 000, and better still from 20 000 to 80 000.

As examples of photodimerizable polymers (P) according to the present invention, mention may notably be made of the mPEG-PVA-SbQ polymers of the following formulae. The molar percentages of each unit (or pendant group) may be varied:

TABLE 1

| | |
|---|---|
| 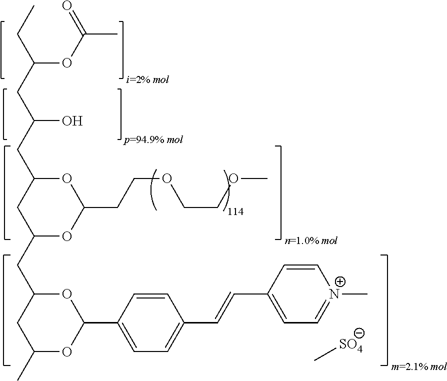 | Compound 1 |
| 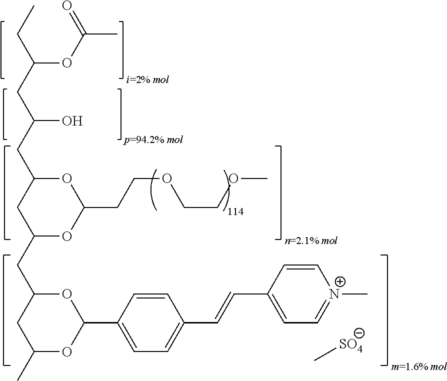 | Compound 2 |
| 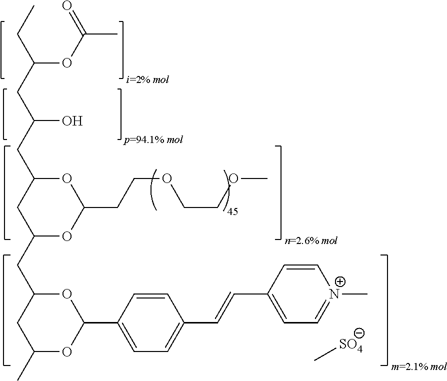 | Compound 3 |
| 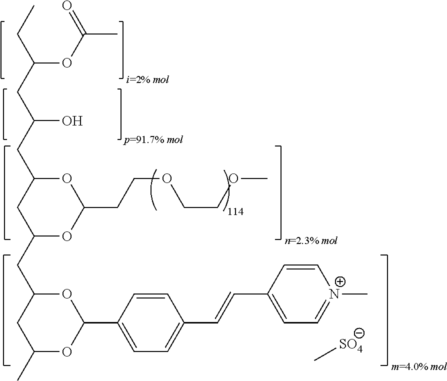 | Compound 4 |

The photodimerizable polymer(s) (P) according to the present invention may optionally also include one or more hydrophobic pendant groups.

As hydrophobic pendant groups that may be used according to the present invention, mention may notably be made of:
saturated or unsaturated (C₁-C₃₀)alkyl groups, optionally substituted and/or interrupted with one or more heteroatoms,
alkenyl groups,
aryl groups such as phenyl, pyridyl, furyl, indolyl, benzofuryl, thiophenyl, imizadolyl, oxazolyl, thiazolyl, pyrazinyl or pyrimidinyl;
fluorinated groups such as fluorocarbon groups such as —CF₃, —CHF₂, —OCF₃, —SCF₃, CF₃C(O)—,
silicone groups such as —SiRₐR_{b}R_{c} such as —Si(CH₃)₃, polydimethylsiloxane-PDMS, —Si(OR)₃, PDMS α,ω diaminopropyl, PDMS α,ω dihydroxyalkyl, PDMS α,ω dicarboxyalkyl, with Rₐ, R_{b} and R_{c}, which may be identical or different, representing a (C₁-C₈)alkyl group which is optionally interrupted and/or terminated with one or more non-contiguous heteroatoms such as O or S, and R representing a (C₁-C₆)alkyl group.

Preferably, when they are present, the pendant hydrophobic group(s) represent a (C₂-C₂₂)alkyl group, more preferentially a (C₃-C₁₆)alkyl group and better still a methyl, octyl or phenyl group.

### Composition

The present invention also relates to a composition comprising one or more photodimerizable polymers (P) as defined previously.

According to a first variant of the invention, the composition comprises a single photodimerizable polymer (P) comprising several photodimerizable pendant groups of identical or different nature.

According to another variant of the invention, the composition comprises a mixture of photodimerizable polymers (P) comprising one or more photodimerizable pendant groups of different nature.

Consequently, the reactions may take place between two photodimerizable groups that may or may not be of the same chemical nature. The activated double bonds may react with another double bond of the same chemical nature or may react with another double bond of different chemical nature.

The total content of the photodimerizable polymer(s) (P), present in the composition according to the invention, preferably ranges from 0.01% to 50% by weight, more preferentially from 0.1% to 25% by weight, even more preferentially from 1% to 20% by weight, better still from 5.5% to 20% by weight, and even better from 6% to 15% by weight, relative to the total weight of the composition.

The composition according to the present invention may optionally also comprise one or more photosensitizers.

For the purposes of the present invention, the term “photosensitizer” means an ingredient which modifies the irradiation wavelength, thereby triggering the photodimerization reaction.

For example, the photodimerization of dimethylmaleimide groups is triggered by irradiation centred on the wavelength range from 270 to 300 nm. In the presence of a photosensitizer such as thioxanthone, photodimerization becomes effective with irradiation centred on the wavelength range from 360 to 430 nm.

Among the photosensitizers that may be used according to the invention, mention may notably be made of thioxanthone, rose Bengal, phloxine, eosin, erythrosine, fluorescein, acriflavine, thionine, riboflavin, proflavine, chlorophylls, haematoporphyrin, methylene blue and mixtures thereof.

The total content of the photosensitizer(s), when they are present in the composition of the invention, preferably ranges from 0.00001% to 5% by weight relative to the total weight of the composition.

According to another embodiment, the composition according to the present invention is preferably aqueous.

The term “aqueous composition” according to the present invention means a composition in which the total water content is greater than or equal to 20% by weight, relative to the weight of the composition. Preferably, the total water content is greater than or equal to 30% by weight, more preferentially greater than or equal to 40% by weight of water, and better still greater than or equal to 50% by weight, relative to the total weight of the composition. Advantageously, the total water content ranges from 40% to 99% by weight, and preferably 50% to 95% by weight, relative to the total weight of the composition.

Besides water, the composition according to this embodiment may optionally comprise one or more organic solvents.

Preferably, the organic solvent(s) are chosen from linear or branched monoalcohols having from 1 to 8 carbon atoms and more preferentially from 1 to 4 carbon atoms, polyols, polyethylene glycols, aromatic alcohols and mixtures thereof.

As examples of organic solvents that may be used according to the invention, mention may notably be made of ethanol, propanol, butanol, isopropanol, isobutanol, propylene glycol, dipropylene glycol, isoprene glycol, butylene glycol, pentylene glycol, hexylene glycol, glycerol, sorbitol, benzyl alcohol and phenoxyethanol, and mixtures thereof.

The organic solvent(s) that may be used according to the invention may be chosen from linear or branched monoalcohols containing from 1 to 4 carbon atoms, and mixtures thereof, preferably from ethanol, propanol, butanol, isopropanol, isobutanol, and mixtures thereof.

The pH of the composition according to the present invention preferably ranges from 3 to 9, and more preferentially from 4 to 8.

The composition according to the present invention may also optionally comprise one or more additional compounds, other than the ingredients of the invention and among which mention may be made of fatty substances, cationic, anionic, nonionic, amphoteric or zwitterionic surfactants, cationic, anionic, nonionic or amphoteric polymers or mixtures thereof, antidandruff agents, antiseborrhea agents, agents for preventing hair loss and/or for promoting hair regrowth, vitamins and provitamins including panthenol, sunscreens, sequestrants, plasticizers, solubilizers, acidifying agents, mineral or organic thickeners, notably polymeric thickeners, antioxidants, hydroxy acids, fragrances, preserving agents and ceramides, and mixtures thereof.

Needless to say, a person skilled in the art will take care to select this or these optional additional compound(s) such that the advantageous properties intrinsically associated with the composition according to the invention are not, or are not substantially, adversely affected by the envisaged addition(s).

The above additional compounds may generally be present in an amount, for each of them, of between 0 and 20% by weight relative to the total weight of the composition.

The composition according to the invention is advantageously a cosmetic composition, and preferably a hair composition, such as a shampoo, a conditioner, a mask or any other presentation form that is conventional in the hair field.

The composition may be in optionally thickened liquid form, cream or paste form or in solid form such as a powder or a film.

### Cosmetic Treatment Process

A subject of the present invention is also a process for the cosmetic treatment of keratin materials, in particular keratin fibres, preferably human keratin fibres such as the hair, comprising:
(a) a step of applying to said keratin materials one or more photodimerizable polymers (P), as defined previously,
(b) followed by an optional step of drying said keratin materials, for example by means of a hair dryer,
(c) followed by a step of exposing said keratin materials to natural or artificial, preferably artificial, light radiation.

Preferably, the photodimerizable polymer(s) (P) used in the process of the invention are introduced into a composition as defined previously. According to this embodiment, the process of the invention comprises:
(a) a step of applying to said keratin materials a composition comprising one or more photodimerizable polymers (P), as defined previously,
(b) followed by an optional step of drying said keratin materials, for example by means of a hair dryer,
(c) followed by a step of exposing said keratin materials to natural or artificial, preferably artificial, light radiation.

Preferably, the process of the invention comprises a drying step (b) between step (a) of applying the photodimerizable polymer(s) and step (c) of exposure to light radiation.

The photodimerizable polymer(s) (P) or the composition used in the process of the invention may be applied to dry or wet, preferably wet, keratin materials.

Preferably, the composition used in the process of the invention is applied to the keratin materials in an amount ranging from 0.01 to 10 grams, more preferentially from 0.1 to 5 grams, and better still from 0.3 to 3 grams of composition per gram of keratin materials.

After application, the photodimerizable polymer(s) (P) or the composition are optionally left to stand on the keratin materials. In other words, after applying the photodimerizable polymer(s) (P) or the composition, a leave-on time may or may not be observed before the possible drying step (b).

The application of the photodimerizable polymer(s) (P) or of the composition is preferably directly followed by the drying step (b), which is itself followed by step (c) of exposing the keratin materials to natural or artificial light radiation, without any intermediate leave-on time.

According to a embodiment of the invention, the light radiation is natural.

For the purposes of the invention, the term “natural light radiation” means light radiation of natural daylight (generated by the sun). The time of exposure to the ambient light may range more particularly from 10 seconds to 30 minutes and notably from 2 to 15 minutes.

According to a preferred embodiment of the invention, the light radiation is artificial, and may or may not be continuous.

For the purposes of the invention, the term “artificial light radiation” means light radiation of artificial light, other than natural daylight (generated by the sun). The time of exposure to said artificial light may range from 1 second to 20 minutes and in particular from 1 second to 1 minute.

Preferably, the artificial light radiation is generated using a device chosen from arc lamps such as xenon lamps and mercury lamps, fluorescent lamps, incandescent lamps such as halogen lamps, light-emitting diodes (LED), organic light-emitting diodes (OLED) and lasers.

Mention may be made, for example, of goLITE BLU from the company Philips, the lamp Energylight HF 3319/01 from the company Philips, the lamps Dayvia White and Messa from the company Solvital, the lamp Lumino Plus from the company Lanaform, the lamp Medibeam from the company Medibeam, the lamp M-LED 01 from the company Meimed, the lamp Lifemax Light Pod from the company Lifemax, the lamp Lite-Pad from the company Reicorp, the lamps Omnilux Clear-U and New-U from the company Omnilux, the 1000 W xenon arc lamp from the company Lot-Oriel and the lamp Camag Box 3 (4×8 W) from the company Camag.

The artificial light source may emit radiation in the visible range and/or radiation in the UV range. The artificial light emitted may or may not be monochromatic.

The artificial light radiation is preferably produced at a wavelength between 280 nm and 700 nm and more preferentially between 300 nm and 500 nm.

Advantageously, the crosslinking is initiated by simple illumination without the need for a photoinitiator.

Preferably, the source of the artificial radiation emits an energy of between 1 and 50 W/cm² of keratin material, the exposure times being adapted accordingly.

The crosslinking may take place with a reduced light intensity: the lighting system may produce, for example, light intensity of between 500 mJ/cm² and 10 J/cm² of keratin material.

The twofold characteristic of the absence of a photoinitiator and the relatively low light intensity is particularly advantageous since it makes it possible to limit the harmful effects of aggressive initiators or of prolonged exposure to intense light, in particular in the UV wavelengths.

A person skilled in the art will be capable of adapting the illumination characteristics, notably in terms of exposure time and of radiation wavelengths, with regard to the nature of the photodimerizable polymer(s) (P) used.

On conclusion of the process of the invention, the keratin materials may optionally be washed with a shampoo or rinsed with water. They may also be dried, for example with a hair dryer, or left to dry.

For the purposes of the present invention, the term “keratin materials” preferably means human keratin materials such as the skin, the nails, the lips, the eyelashes, the eyebrows and the hair. More particularly, keratin materials are keratin fibres, preferably human keratin fibres, and better still the hair.

The examples that follow serve to illustrate the invention without, however, being limiting in nature.

### EXAMPLES

### Example I: Synthesis of a Photocrosslinkable mPEG-PVA-SbQ Polymer According to the Invention

A photocrosslinkable mPEG-PVA-SbQ polymer according to the invention (formula below) was synthesized according to the following protocol: synthesis of the PVA-SbQ polymer followed by a step of grafting the PEG groups.

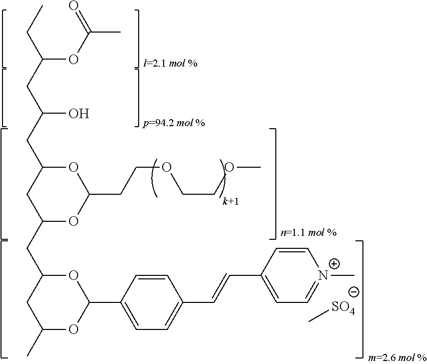

First step: synthesis of the PVA-SbQ polymer according to the publication: “Synthesis of hydrophobized and photo-crosslinkable pre-polymer based on polyvinyl alcohol” in Reactive & Functional Polymer, 1995, 28, 55-60.

The PVA-SbQ polymer was synthesized according to the following synthetic reaction

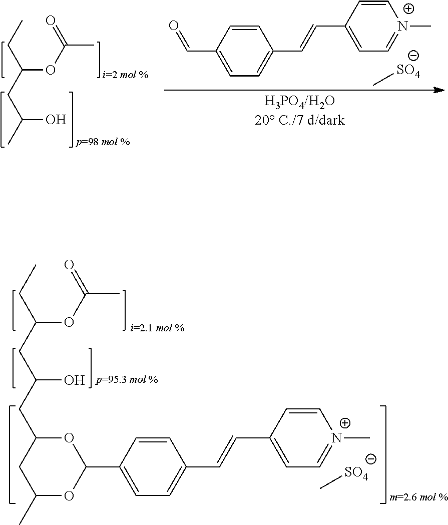

The amounts of the reaction compounds used in this synthesis are expressed (in grams) in the table below:

TABLE 2

| Solutions | Compounds | Amount (g) |
|---|---|---|
| PVA solution | PVA, Mowiol 4-98 | 115.42 |
| | 85% H₃PO₄ | 23.26 |
| | H₂O | 2242 |
| SbQ-4 solution | SbQ-4 | 29.20 |
| | H₂O | 115.50 |

115.42 g of polyvinyl alcohol (PVA, Mowiol 4-98) were dissolved in 2242 g of water with stirring (80 rpm) at 90° C. The reaction medium was brought to 20° C. and left stirring (20 rpm) overnight. 23.26 g of phosphoric acid (85%) were then slowly added to the medium. A light shield (aluminium foil) was then placed around the reactor.

In parallel, 29.20 g of SbQ-4 were dissolved in 115.5 g of water, in the absence of light, before being added to the reaction medium (i.e. the above PVA solution). The medium was then left stirring (100 rpm) for 5 days, protected from light.

After 3 days, a sample (15 g) of the reaction medium was taken and precipitated from 120 g of acetone. The yellow solid thus obtained was then washed 4 times with acetone before being dried under vacuum. NMR analysis confirmed that the product obtained was the desired PVA-SbQ polymer.

The remaining reaction medium was used for the second step below, without prior purification.

Second step: grafting of PEG groups

The mPEG-PVA-SbQ polymer was synthesized according to the following synthetic reaction

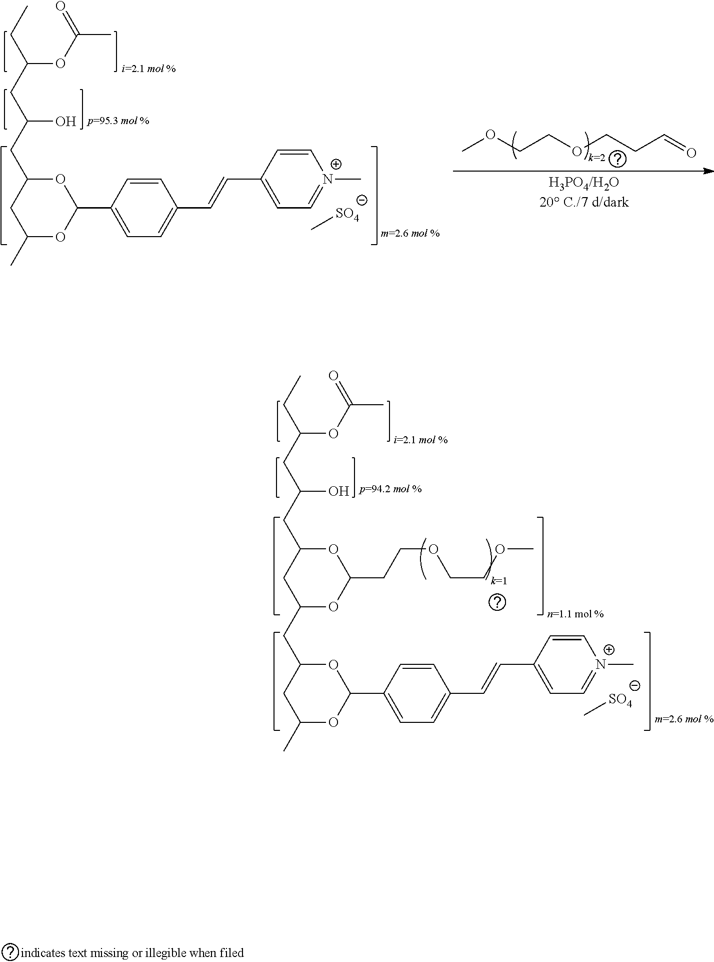

The amounts of the reaction compounds used in this synthesis are expressed (in grams) in the table below:

TABLE 3

| | Amount (g) |
|---|---|
| PVA-SbQ polymer synthesized in the first step | 1499.72 |
| mPEG5K propionaldehyde | 120.93 |
| 85% H₃PO₄ | 5.20 |
| H₂O | 514.30 |

120.93 g of mPEG5K propionaldehyde were dissolved in water and phosphoric acid, in the absence of light. The solution was heated at 50° C. for 1 h before being cooled to room temperature and degassed.

The PVA-SbQ previously obtained was then added to the mixture, with stirring (300 rpm) and in the absence of light. After 7 days, 10 mL of the reaction medium were precipitated from 50 mL of an MEK/acetone (80/20) mixture. The solid obtained was then dissolved in 15 mL of methanol before being precipitated again from 32 mL of MEK/acetone (80/20) mixture.

The solid product thus obtained was dried under vacuum at room temperature to give the desired mPEG-PVA-SbQ polymer.

### Example II: Evaluation of the Disentangling Properties

a. Preparation of the Formulations

Formulation A according to the present invention and comparative formulations B1 and B2 were prepared from the ingredients mentioned in the table below, expressed as percentages of active material.

TABLE 4

| | A | B1 | B2 |
|---|---|---|---|
| | (invention) | (comparative) | (comparative) |
| mPEG-PVA-SbQ | 6% | — | — |
| (obtained | | | |
| in Example I) | | | |
| PVA-SbQ | — | 6% | — |
| Propyl-PVA-SbQ | — | — | 6% |
| Distilled water | qs 100% | qs 100% | qs 100% |

b. Protocol

Formulations A, B1 and B2 thus obtained were applied to 2.5 g locks of 27 cm long sensitized hair (AS20) and moistened at a rate of 0.5 g of formulation per gram of hair. Blow-drying was then performed on each of the locks using a 30 cm diameter round brush and a hair dryer.

During the blow-drying, the tangential force of the brush as it passed through the lock was measured for each of the locks using a Lloyd machine (Ametek LS1 measuring machine) for 30 seconds.

c. Results

The sums of the tensile forces measured for 30 seconds for each of the locks are expressed in the table below.

TABLE 5

| | | Force (N) measured |
|---|---|---|
| | Force (N) measured at | after five shampoo |
| | t₀ | washes |
| A (invention) | 479 | 633 |
| B1 (comparative) | 1453 | 1266 |
| B2 (comparative) | 1208 | 1094 |
| Control - sensitized but | 2015 | — |
| untreated lock | | |

The results obtained above show that hair treated with the formulation of the invention, comprising a photodimerizable polymer (P) comprising at least one polyoxyalkylene pendant group (mPEG-PVA-SbQ), has better disentangling properties than hair treated with the comparative formulations, not containing the particular polymer (P). Specifically, the force of resistance to the passage of the brush, measured after applying formulation A, is significantly lower than the force measured for the comparative formulations, which makes brushing much easier.

### Example III: Compositions According to the Invention

Compositions A1 and A2 according to the present invention were prepared from the ingredients mentioned in the table below, expressed as weight percentages of active material.

TABLE 6

| | A1 | A2 |
|---|---|---|
| 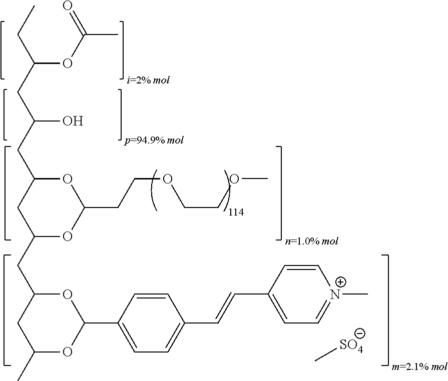 | 6 | 6 |
| Xanthan gum | 1.5 | 2 |
| Mixture of amino silicones (Belsil ADM | — | 0.074 |
| LOG1 from Wacker) | | |
| Water | qs 100% | qs 100% |

Compositions A1 and A2 thus obtained give the hair improved conditioning properties.

### Example IV: Evaluation of the Disentangling Properties and of the Flexibility of the Hair

a. Preparation of the Formulations

Formulation A3 according to the present invention and comparative formulations C₁, C₂, C₃ and C₄ were prepared from the ingredients mentioned in the table below, expressed as percentages of active material.

TABLE 7

| | A3 | C1 | C2 | C3 | C4 |
|---|---|---|---|---|---|
| | (inv.) | (Comp.) | (Comp.) | (Comp.) | (Comp.) |
| mPEG-PVA- | 5 | — | — | — | — |
| SbQ (obtained | | | | | |
| in Example I) | | | | | |
| PVA-SbQ | — | 5 | — | 3.24 | 5 |
| PEG-90 | — | — | 5 | 1.76 | 2.72 |
| Distilled water | qs | qs | qs | qs | qs |
| | 100% | 100% | 100% | 100% | 100% |

b. Protocol

Formulations A3, C₁, C₂, C₃ and C₄ thus obtained were applied to 2.5 g locks of 25 cm long sensitized hair (AS20) and moistened at a rate of 0.4 g of formulation per gram of hair. Blow-drying was then performed on each of the locks using a 30 cm diameter round brush and a hair dryer.

The locks then underwent an artificial light irradiation at a wavelength of 405 nm.

Then, the locks are washed 5 times with a standard shampoo (DOP®), blow-dried, washed 5 times again with a standard shampoo (DOP®) and blow-dried.

10 experts evaluated by touch and naked eye, the flexibility of treated hair locks compared to untreated hair locks, and gave a score between 1 and 10; wherein 1=the hair is similar to untreated hair and 10=the flexibility of the hair has been particularly improved compared to untreated hair.

The disentangling properties have also been evaluated. The treated locks were plunged completely into a beaker (800 ml) of distilled water 3 times to entangle the hair, and then wrung out with fingers. A comb is then passed along the entire length of the treated lock and the distance the comb has run from the root to the tip before being stopped by a knot has been measured.

c. Results

The results are summarized in the tables below.

TABLE 8
Flexibility

| Formulations | Average score |
|---|---|
| A3 (inv.) | 10 ± 0 |
| C1 (Comp.) | 2.7 ± 0.5 |
| C2 (Comp.) | 2.3 ± 0.5 |
| C3 (Comp.) | 5.7 ± 0.5 |
| C4 (Comp.) | 5.3 ± 0.5 |

TABLE 9
Disentangling properties

| Formulations | Distance (in cm) before being stopped |
|---|---|
| A3 (inv.) | The comb has not been stopped |
| C1 (Comp.) | 10 cm |
| C2 (Comp.) | 13 cm |
| C3 (Comp.) | 20 cm |
| C4 (Comp.) | 19 cm |

The results obtained above show that the formulation A3 of the invention, comprising a photodimerizable polymer (P) comprising at least one polyoxyalkylene pendant group (mPEG-PVA-SbQ), provides much better flexibility and disentangling properties to the hair than the comparative formulations which contain either the PVA-SbQ polymer alone or the PEG polymer alone or a mixture of the PVA-SbQ polymer and the PEG polymer.

1. Photodimerizable polymer (P) comprising at least one photodimerizable pendant group and at least one polyoxyalkylene pendant group, wherein the photodimerizable pendant group(s) are chosen from monovalent radicals of formulae (I) and (II) below: 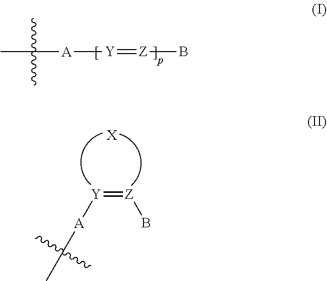
and also the geometrical isomers thereof,
in which formulae (I) and (II):
Y and Z denote, independently of each other, a nitrogen atom or a C(R) group with R representing a hydrogen atom or a (C₁-C₄)alkyl group such as methyl;
A represents a bond or a divalent group chosen from (C₁-C₅)alkylene, arylene, heteroarylene, cycloalkylene, heterocycloalkylene, (thio)carbonyl and (C₂-C₅)alkenylene radicals and combinations thereof;
B represents a monovalent group chosen from (C₁-C₅)alkyl radicals, aryl radicals, optionally cationic heteroaryl radicals, cycloalkyl radicals, optionally cationic heterocycloalkyl radicals, (thio)carbonyl radicals and (C₂-C₅)alkenyl radicals and combinations thereof;
X represents a divalent group chosen from (C₂-C₅)alkylene, arylene, heteroarylene, cycloalkylene, heterocycloalkylene, (thio)carbonyl and (C₂-C₅)alkenylene radicals and combinations thereof;
p represents an integer between 1 and 5 inclusive, in particular between 1 and 3; preferably, p is equal to 1; 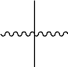
represents the bond which connects the part of the monovalent radical to the rest of the molecule; and
each of the groups mentioned may optionally be substituted with one or more halogen atoms or groups chosen from the following: (C₁-C₆)alkyl, hydroxyl, amino, (di)(C₁-C₆)alkylamino, phenyl, carboxyl, (C₁-C₆)alkoxy, (C₁-C₆)alkoxy(thio)carbonyl, hydrogeno(thio)carbonyl, sulfonato R—O—S(O)₂— or R—S(O)₂—O⁻, amide RR′N—C(O)— or R—C(O)—N(R′)— or acyl R—C(O)—, ammonium R′R″N⁺— with R, R′ and R″, which may be identical or different, representing a hydrogen atom or a (C₁-C₄)alkyl group.

2. Polymer according to claim 1, characterized in that the photodimerizable pendant group(s) are chosen from monovalent radicals of the following compounds:
stilbene,
styrylpyridinium (stilbazolium) of formulae (A1) and (A2) below, and the geometrical isomers thereof: 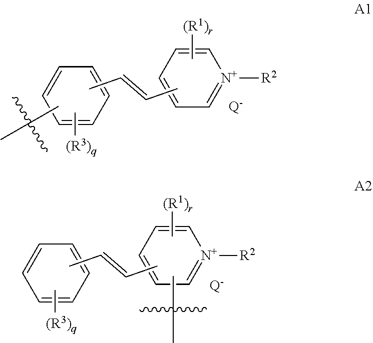
in which:
R¹ and R³, which may be identical or different, represent a halogen atom or a (C₁-C₆)alkyl group; or else two contiguous R¹ or R³ groups form, together with the carbon atoms bearing them, a benzo group;
R² represents a hydrogen atom, a (C₁-C₆)alkyl group optionally substituted with one or more halogen atoms such as chlorine or hydroxyl; preferably, R² represents a (C₁-C₆)alkyl group such as methyl, ethyl or propyl;
q and r represent an integer between 0 and 4 inclusive; and
Q⁻ represents an anionic counter ion preferably chosen from halide ions such as chlorides, bromides and iodides, perchlorates, tetrafluoroborates, methyl sulfate, phosphates, sulfates, methanesulfonates, p-toluenesulfonate; and 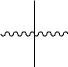
represents the bond which connects the part of the monovalent radical to the rest of the molecule, it being understood that the pendant group A2 can be connected to the rest of the molecule via R²;
preferably, the 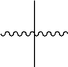
bond is on the phenyl in the para position of the styryl group on A1 or connected to the rest of the molecule via R² on A2; preferentially, the styryl group of A1 and A2 is in the para position of the pyridinium group;
styrylazolium of formulae (B1 and B2) below, and the geometrical isomers thereof: 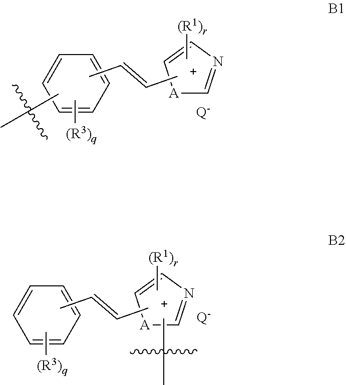
in which:
A represents a sulfur atom, an oxygen atom, or a group NR₂ or C(R²)₂; and 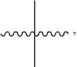
Q⁻, r, q, R¹, R² and R³ being as defined previously,
preferably, the 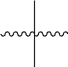
bond is on the phenyl in the para position of the stryryl group,
styrylpyrazine,
chalcone,
(thio)cinnamate and (thio)cinnamamide,
maleimide,
(thio)coumarin,
thymine,
uracil,
butadiene,
anthracene,
pyridone,
pyrrolizinone,
acridizinium salts,
furanone,
phenylbenzoxazole, and
derivatives thereof.

3. Polymer according to claim 1, characterized in that the polyoxyalkylene pendant group(s) comprise one or more polyoxyalkylene groups chosen from the groups of the following formula:
—[O—X]ₙ—OR
in which, X represents an alkylene group containing from 2 to 3 carbon atoms, preferably 2 carbon atoms, n denotes an integer ranging from 10 to 1000, preferably from 20 to 200, and more preferentially from 25 to 150, and R represents a linear or branched alkyl chain containing from 1 to 6 carbon atoms.

4. Polymer according to claim 1, characterized in that it has a natural or synthetic backbone chosen from polysaccharides, poly(vinyl) polymers and polydiorganosiloxanes, preferably from polyvinyl alcohols and poly(vinyl acetate)s, which are preferably partially hydrolysed.

5. Polymer according to claim 1, characterized in that the total amount of the photodimerizable pendant group(s) ranges from 0.5 to 6 mol %, and preferably from 1 to 5 mol %, relative to the total molar weight of the polymer.

6. Polymer according to claim 1, characterized in that the total amount of the polyoxyalkylene pendant group(s) ranges from 0.1 to 5 mol % and preferably from 0.5 to 3 mol %, relative to the total molar weight of the polymer.

7. Composition comprising one or more photodimerizable polymers (P) as defined according to claim 1.

8. Composition according to claim 7, characterized in that the total amount of the photodimerizable polymer(s) (P) ranges from 0.01% to 50% by weight, preferably from 0.1% to 25% by weight and better still from 1% to 20% by weight, relative to the total weight of the composition.

9. Composition according to claim 7, characterized in that it further comprises water, preferably in a total content of greater than or equal to 30% by weight, more preferentially greater than or equal to 40% by weight, and better still greater than or equal to 50% by weight, relative to the total weight of the composition.

10. Cosmetic process for treating keratin materials, in particular keratin fibres, preferably human keratin fibres such as the hair, comprising:
(a) a step of applying to said keratin materials one or more photodimerizable polymers (P), as defined according to claim 1,
(b) followed by an optional drying step,
(c) followed by a step of exposing said keratin materials to natural or artificial light radiation.

11. Process according to claim 10, characterized in that the light radiation is artificial; preferably, the artificial light radiation is produced at a wavelength of between 280 nm and 700 nm and more preferentially between 300 nm and 500 nm.
